(19) United States
(12) Patent Application Publication
(10) Pub. No.: US 2019/0350839 A1
(43) Pub. Date: Nov. 21, 2019

(54) POLYP LIFTING COMPOSITIONS AND METHODS FOR USE

(71) Applicants: IMPERIUM MEDICAL SYSTEMS LIMITED, Thirsk (GB); IMPERIAL COLLEGE INNOVATIONS LIMITED, London (GB)

(72) Inventors: Daniel Edmondson, York (GB); Brian Saunders, London (GB)

(73) Assignees: IMPERIUM MEDICAL SYSTEMS LIMITED, Thirsk (GB); IMPERIAL COLLEGE INNOVATIONS LIMITED, London (GB)

(21) Appl. No.: 16/479,368

(22) PCT Filed: Jan. 19, 2018
(86) PCT No.: PCT/GB2018/050172

§ 371 Date: Jul. 19, 2019

(30) Foreign Application Priority Data
Jan. 19, 2017 (GB) 1700943.2
Apr. 26, 2017 (GB) 1706647.3

(51) Int. Cl. A61K 9/00; A61K 31/728; A61K 9/08; A61K 47/02; A61K 49/00; A61B 10/04

(52) U.S. Cl. CPC A61K 9/0019; A61K 31/728; A61K 9/08; A61B 2010/045; A61K 49/006; A61B 10/04; A61K 47/02

(57) ABSTRACT

A composition for aiding the removal of polyps, in the form of a submucosal injectable formulation, comprising one or more salts of hyaluronic acid, saline solution and indigo carmine, but not including a substance exhibiting an angiotonic effect such as epinephrine, norepinephrine, or isoproterenol is described. Further described are a method for removing a polyp from a mucous membrane, the composition for use in endoscopic procedures and a kit for use in an endoscopic procedure.

### FIELD OF THE INVENTION

The present invention relates to compositions for aiding the removal of polyps, methods for removing polyps and to use of the compositions in the method of removing polyps.

### BACKGROUND OF THE INVENTION

A polyp is an abnormal growth of tissue, typically found in the colon, stomach, nose, ear, sinus, urinary bladder or uterus, which projects from the mucous membrane. A polyp may be attached to the surface via a narrow elongated stalk (“pedunculated”) or may lie flat against the surface (“sessile”). Some polyps are tumours and may be benign or premalignant and/or concurrent with a malignancy. Therefore, polyps are typically identified and removed for biopsy.

In therapeutic endoscopy, such as colonoscopy, polyps for removal are identified and removed by “snares” for biopsy. In this process, a wire lasso is looped around the polyp and then drawn closed, incising the abnormal tissue growth from the mucosa. The bowel wall is relatively thin and to avoid perforation during polypectomy the wire snare must transect a plane above the main muscle, the submucosa. Sessile or flat polyps that grow around the bowel wall are challenging to remove as they are difficult to grasp with the snare. Enlarging the submucosal space below sessile or flat polyps enables grasping with the snare or cutting with endoscopic knives to allow a complete polyp excision either in a single piece or piecemeal.

Compositions as discussed by U.S. Pat. No. 6,319,260 are known; however, such compositions including adrenaline (epinephrine) may be disadvantageous, due to systemic cardiovascular effects and masking of bleeding vessels that require coagulation post-polypectomy

Accordingly, there exists a need to provide injection solutions that are easy to use and achieve a sustained submucosal lift with clear definition of the submucosal space without systemic toxicity.

### SUMMARY OF THE INVENTION

In view of the foregoing, a first aspect of the invention is to provide a composition for aiding the removal of polyps wherein the composition is a sterile submucosal suitable for easy to injection formulation comprising;
one or more salts of hyaluronic acid,
saline solution, and
indigo carmine,
but not including a substance exhibiting an angiotonic effect such as epinephrine, norepinephrine, or isoproterenol.

Suitably, the hylauranate provided by the one or more salts of hyaluronic acid allows the formulation to be easily injected.

The saline may have any suitable concentration. A standard concentration is 0.1%.

Suitably, the composition may comprise a preservative, for example a preservative to maintain the colour of the composition, as provided by indigo carmine.

According to embodiments of the invention the composition for aiding the removal of polyps, can be provided in the form of a submucosal injectable formulation, consisting essentially of, or consisting of, one or more salts of hyaluronic acid, saline solution and indigo carmine. In embodiments saline and the one or more salts of hyaluronic acid can be Sigmavisc™ (sodium hyaluronic and saline). Suitably the salt of hyaluronic acid may be sodium hyaluronate.

In embodiments the composition can be provided in a sterile vial capable of holding 5 ml to 15 ml, suitably 8 ml to 12 ml, suitably 10 ml of the composition.

According to a second aspect of the invention, there is provided a method for removing a polyp from a mucous membrane comprising the steps of:

injecting a composition of the first aspect of the invention below the polyp; and resecting the polyp from the mucosa. Suitably resecting of the polyp may be for biopsy.

According to a third aspect of the present invention, there is provided a composition according to the first aspect of the invention for use in endoscopic procedures.

According to a fourth aspect of the present invention there is provided a kit for use in an endoscopic procedure, said kit comprising a composition according to the first aspect of the invention, an endoscopic injection needle, optionally a syringe and instructions for use thereof.

The present inventors have determined that adrenaline is not required to allow a suitable lift time for the polyp to allow the method of the invention. Further, they have determined that a sterile pre-mixed composition, as described in the first aspect of the invention, which can be drawn up through a syringe, is advantageous, as it ensures an appropriate amount of indigo carmine is provided for staining and subsequent visualisation. Non-sterile contemporaneous preparation of such a composition or solution risks insufficient or too much stain being provided as the quantity of stain is difficult to determine from the intensity of the colour during mixing. Further contemporaneous preparation is likely to be undertaken under non-sterile conditions.

### DETAILED DESCRIPTION

The “hyaluronic acid” according to the invention may be, for example, a high molecular weight polysaccharide containing glucuronic acid and N-acetylglucosamine as repeat units. Suitably, it may also include hyaluronic acid derivatives in which a portion of the hyaluronic acid has been derivatised. It can be, for example, a polymer of two saccharides comprising glucuronic acid and N-acetylglucosamine, with an average molecular weight of from 50,000 to 13,000,000 daltons.

In embodiments of the invention, hyaluronic acid can have an average molecular weight of about 200,000 to 4,000,000, suitably about 600,000 to 1,200,000.

Suitable salts of hyaluronic acid may include, but are not limited to, for example, sodium, potassium, calcium, aluminium, zinc, iron, ammonium, and tetrabutylammonium salts.

In embodiments of the invention the pharmaceutically acceptable salt of hyaluronic acid can be sodium. In embodiments the hyaluronic acid can be sodium hyaluronate EC number 618 620-0, CAS number 9067 32-7 with a molecular weight of 1000 to 5000000g/mole.

Hyaluronic acid and its pharmaceutically acceptable salts can be produced using various known methods, such as a method by extraction from biological sources such as the rooster comb and pig subcutaneous tissue, via a biofermentation method, or by purchasing commercial products. Suitably the hyaluronic acid may be obtained by fermentation of Streptococcus as known in the art.

Indigo carmine, otherwise known as 5,5′-indigodisulfonic acid sodium salt is approved for use as a food and a pharmaceutical colourant in the U.S and Europe. It has the E number E132. Indigo carmine is a commercially available product and thus can be obtained from several suppliers.

Whilst other dyes can be used, indigo carmine is advantageous as it does not interact with DNA. As following application of the composition the stain will remain in the body, it is preferable that the stain does not interact with DNA as this could lead to tumours. Further, indigo carmine is advantageous as it is visible as an ‘electric blue’ stain under endoscopic light. This aids in defining the submucosal space and the edges of sessile or flat polyps.

According to an embodiment of the invention, gastrointestinal endoscopy is preferably performed in the esophagous, stomach and/or small intestine (duodenum, jejunum, ileum), in the large intenstine, suitably the caecum, in the colon (e.g. ascending, transverse and descending), in the sigmoid colon and/or in the rectum. Suitably a saline solution, one or more salts of hyaluronic acid and indigo carmine can be used in endoscopic procedures according to the invention including endoscopic resection as performed during a gastrointestinal endoscopy, including endoscopic biopsy, a polypectomy, an endoscopic mucosal resection (EMR) and/or an endoscopic submucosal dissection (ESD).

According to the invention the term “polyps” includes pseudo-polyps and flat polyps.

Suitably, the composition of the first aspect of the invention may consist essentially of saline solution, one or more salts of hyaluronic acid and indigo carmine. Suitably, “consisting essentially” allows the presence of other components, provided the essential characteristics of the claimed composition are not materially affected. In particular, the composition should not include components which exhibit angiogenic effect. Suitably, compositions consisting essentially of one or more salts of hyaluronic acid, saline solution and indigo carmine may also include preservative to maintain the colour of the composition. In embodiments, the composition of the present invention can be a transparent, slightly viscous, solution particularly indicated for submucosal injection in polypectomy.

Suitably, the composition of the first aspect of the invention may consist of saline solution, one or more salts of hyaluronic acid and indigo carmine.

In embodiments the hyaluronic acid and the indigo carmine component of the composition according to the present invention are suitably formulated as an aqueous solution.

Suitably, the composition may be formulated as an injectable formulation. In order to formulate the constituents of the composition as an injectable formulation it may be dissolved in a desired concentration in a saline solution. In embodiments the saline solution is a physiological saline or phosphate-buffered physiological saline. Suitably the formulation may be adjusted to a desired pH by adding acid or base as needed.

When administering the composition according to the present invention as a submucosal injectable formulation, the concentration of sodium hyaluronate can be the range of 0.1 to 1% weight/volume (w/v), suitably 0.24 w/v to 0.40% w/v.

In embodiments of the invention, the concentration of sodium hyaluronate can be in the range 0.3-0.35%% w/v, such as approximately 0.31-0.32%% w/v.

In embodiments, the concentration of indigo carmine can be in the range of 0.001 to 1% weight/volume (w/v). As indigo carmine does not interact with DNA, it can be used in any amount to achieve a ‘marker’ effect. Thus, in one embodiment, the concentration of indigo carmine can be in the range of 0.1 to 1% weight/volume (w/v), such as in the range 0.29 w/v to 0.48% w/v. In another embodiment, the concentration of indigo carmine can be less than 0.1% weight/volume (w/v) , such as in the range of 0.001 to 0.1% weight/volume (w/v), including in the range of 0.001 to 0.01% weight/volume (w/v) and in the range of 0.01 to 0.1% weight/volume (w/v). Examples include using 0.005, 0.006, 0.007, 0.008, 0.009 and 0.01% weight/volume (w/v).

The amount of indigo carmine used in the present invention, in relation to using 1000g of a saline solution, to achieve the above concentrations can be a suitable number of milligrams in weight, such as in the range 10-100 milligrams of indigo carmine, including approximately 10, 20, 30, 40, 50, 60, 70, 80 , 90 or 100 milligrams.

In embodiments of the invention, the concentration of indigo carmine can be approximately of 0.38%% w/v or 0.009%% w/v.

An injectable formulation according to the present invention can be administered, at 5 ml to 15 ml per administration, suitably 10 ml into the region of the polyp site.

Suitably the composition may be provided pre-mixed as a sterile solution in a 10 ml vial(s). Suitably the sterile solution may be drawn into a syringe directly from the vial for injection.

This administration may be divided into a plurality of times, and the dose can be increased or decreased as appropriate to give a particular optimal dose taking into consideration the physician's instructions, the particular patient, the site of administration, the molecular weight of the hyaluronic acid used and so forth.

### DEFINITIONS

References in the specification to “one embodiment”, “an embodiment”, “in embodiments” and similar indicate that the described embodiment may include a particular aspect, feature, structure or characteristic. Moreover, such phrases may, but do not necessarily, refer to the same embodiment referred to in other portions of the specification. Further, when a particular aspect, feature, structure or characteristic is described in connection with an embodiment, it is within knowledge of a person skilled in the art to affect or connect said aspect, feature, structure or characteristic with other embodiments, whether or not explicitly described.

The singular forms “a”, “an” and “the” include plural references unless the context clearly dictates otherwise. Thus, for example, a reference to “a compound” includes a plurality of such compounds. It is further noted that the claims may be drafted to exclude any optional element. As such, this statement is intended to serve as antecedent basis for the use of exclusive terminology, such as “solely”, “only”, and the like, in connection with the recitation of claims elements or use of a “negative” limitation. The term “and/or” means anyone of the items, any combination of the items, or all the items with which this term is associated.

The terms “comprising”, “having”, “including” and “containing” are to be construed as open-ended terms (i.e. meaning “including, but not limited to”).

The terms “consist essentially of, “consisting essentially of” are to be construed as a semi-closed terms, meaning that no other ingredients which materially affects the basic and novel characteristics (and optionally physiologically acceptable excipients and/or adjuvants) of the invention are included.

Unless indicated otherwise herein, the term “approximately” is intended to include values, e.g. weight percentages, proximate to the recited value that are equivalent in terms of the functionality of the individual ingredient, the composition, or the embodiment. Suitably, the term “approximately” may be ±10% of a stated value.

A person skilled in the art will recognize that, for any and all purposes, particularly in terms of providing a written description, all ranges recited herein also encompass any and all possible sub-ranges and combinations of sub-ranges thereof, as well as the individual values making up the range, particularly integer values. A recited range includes each specific value, integer, decimal, or identity within the range.

The “viscosity” defines the resistance of a liquid or semisolid against flow. The flow of liquids or semisolids is described by viscosity, or, more precisely, by shear viscosity η. The shear viscosity of a fluid expresses its resistance to shearing flows, where adjacent layers move parallel to each other with different speeds. Common units of measurement of viscosity are the pascal-second (Pa s), the poise (P) and cP (centipoises).

The term “endoscopic mucosal resection” (EMR) refers to an endoscopic technique developed for removal of sessile or flat polyps confined to the superficial layers (mucosa and submucosa) of the GI tract.

### EXAMPLES

Hereinafter, examples of the present invention will be described in more detail; however, the present invention is not limited to these examples.

### Example 1

### Polyps Lifting Composition

A solution of 0.315% sodium hyaluronate, 99.676% saline solution and 0.009% indigo carmine was prepared under sterile conditions and provided to a sterile vial.

Suitably the solution in the vial is accessible via a needle attached to a syringe without emptying the vial contents into another container.

### Example 2

### Polyps Lifting Studies

The performance of the composition of the present invention was evaluated in polypectomy in a porcine termination model (in vivo and ex vivo) compared to other existing submucosal solutions.

The safety (muscle viability) and efficacy of the composition of the present invention was assessed in a one-piece (en bloc) cold and hot snare polypectomy including Endoscopic Mucosal Resection—EMR and Endoscopic Submucosal Dissection—ESD techniques.

The submucosal lift and depth, when using the composition of the present invention, was also compared to other existing solutions.

Three submucosal solutions were assessed:

### Normal Saline (NS),

Succinylated Gelatin (SG) (referred to as Gelofuscin in the tables below) and the composition of the present invention (referred to as Deep Blue (DB) in the tables below)
in three polypectomy modalities (cold snare/CS using 9 mm Exacto snare; en bloc-EMR/e-EMR using 14 mm Histolock; and ESD using 2.00 mm BT Flushknife). A designated score was utilised to assess the injection performance of each solution (0 —difficult to inject, 1—easy to inject, 2—very easy to inject).

For each solution, a submucosal cushion was created in separate sites by injecting various volumes: 2 ml for CS, 5 ml for e-EMR and 10 ml for ESD. The degree of the submucosal lift was measured 1 minute after the initial injection using a biopsy forceps. Polypectomy took place using the three techniques and accessories—(Exacto cold snare, histolock resection device, and ESD BT flushknife). In each case the suitable accessory was inserted into the lumen of a colonoscope, the accessory in question was deployed at which point the polyp was resected from the bowel mucosa. During ESD resection, the extra saline amount used was monitored. A designated score was utilised to assess the cutting performance with each solution (0—difficult to cut, 1—easy to cut, 2—very easy to cut), the overall safety provided by each solution during cutting (0—unsafe, 1—very risky, 2—moderately risky, 3—marginally risky, 4—reasonably safe, 5—very safe) and the fluid bleb/cushion sustained by each solution during cutting (0—no cushion, 1—not significant cushion, 2—reasonable cushion, 3—good cushion, 4—very good cushion, 5—excellent cushion).

At the end of the polypectomies, a non-resection assessment was performed comparing the three solutions. 2 ml from each solution was injected within the submucosa and the height of the lift was measured at 5 mins or 10 mins.

Following the colectomy, all resection sites were fixed and one cross-sectional slide was analysed under the light microscope to assess the degree of the submucosal depth and the muscle viability.

On fresh excised specimen, the three solutions were injected (10 ml ) and the height of the submucosal lift was measured blindly in different time intervals in 5, or 20 minutes respectively. After measuring the height (lift), a cross sectional slice was made by a microtome and fixed for light microscope to measure the submucosal depth on the different time intervals (4 slides for 15 minutes and 4 slides for 20 minutes).

Tables 1 to 8 provide the results provided by the porcine termination model studies.

TABLE 1
lifting assessment in vivo

| | Distance Anal verge | | |
|---|---|---|---|
| | 20-25 cm | 30-32 cm | 40-42 cm |
| | Solutions | | |
| | Normal | | |
| Techniques | saline | Gelofuscin | Deep Blue |
| Cold snare | 1 | 0 | 0 |
| En bloc EMR | 1 | 0 | 0 |
| ESD | 2 | 0 | 0 |

0—difficult to inject,
1—ease to inject,
2—very ease to inject

TABLE 2
Extra saline used to sustain lift of ESD in vivo resections

| Extra normal | solution | | |
|---|---|---|---|
| saline | Normal saline | Gelofuscin | Deep Blue |
| Normal saline | 2 ml | 2 ml | 0 ml |

TABLE 3
Lifting height in vivo measurements after 1 min using biopsy forceps

| | Lift height | | |
|---|---|---|---|
| Techniques | Normal saline | Gelofuscin | Deep Blue |
| Cold snare 2 ml | 3 mm | 7 mm | 7 mm |
| En bloc EMR | 3 mm | 5 mm | 7 mm |
| 5 ml | | | |
| ESD | 3 mm | 7 mm | 7 mm |
| 10 ml | | | |

TABLE 4
Snaring assessment in vivo

| | Solutions | | |
|---|---|---|---|
| | Normal | | |
| Techniques | saline | Gelofuscin | Deep Blue |
| Cold snare | 1 | 2 | 1 |
| En bloc EMR | 1 | 1 | 1 |
| ESD | 1 | 1 | 1 |

0—difficult to cut,
1—easy to cut,
2—very easy to cut

TABLE 5
Safety provided by each solution

| | Solutions | | |
|---|---|---|---|
| | Normal | | |
| Techniques | saline | Gelofuscin | Deep Blue |
| ESD | 4 | 4 | 4 |

0—unsafe,
1—very risky,
2—moderately risky,
3—marginally risky,
4—reasonably safe,
5—very safe

TABLE 6
Fluid cushion sustainability

| | Solutions | | |
|---|---|---|---|
| | Normal | | |
| Techniques | saline | Gelofuscin | Deep Blue |
| ESD | 3 | 4 | 4 |

0—no cushion,
1—not significant cushion,
2—reasonable cushion,
3—good cushion,
4—very good cushion,
5—excellent cushion

TABLE 7
Lifting height in vivo measurements

| | Solution 2 ml | | |
|---|---|---|---|
| | Normal | | |
| Time intervals | saline | Gelofuscin | Deep Blue |
| Initial height | 3 mm | 5 mm | 6 mm |
| 5 mins | 1 mm | 2 mm | 4 mm |
| 10 mins | <1 mm | <1 mm | 1 mm |

TABLE 8
Lifting height Ex-vivo measurements

| | Solution 10 ml | | |
|---|---|---|---|
| | Normal | | |
| Time intervals | saline | Gelofuscin | Deep Blue |
| Initial height | 4 mm | 6 mm | 9 mm |
| After 15 mins | 3 mm | 3 mm | 5 mm |
| After 20 mins | 2 mm | 3 mm | 4 mm |

### Example 3

### Endoscopic Procedures

The colon and rectum of the test subject were prepared by flushing with copious amounts of warm tap water via an endoscope to remove any faeces. A Gel point path was then mounted at the anal canal to create a port for the scope passage providing a stable platform access during the operation.

In the animal, all pseudo-polyps for each solution were created from caudal-cranial direction in a longitudinal pattern. The height of the submucosal lift was measured using a closed biopsy forceps after a minute. The pseudo-polyps were then resected applying the three different polypectomy modalities (CS, e-EMR, ESD) as described in the above study design section. This provided three resection sites treated with the composition of the present invention (DS) and sites treated with other existing solutions (NS, and SG). For each of the sites, various volumes were be injected via an endoscopic catheter according to the technique applied (2 ml for CS, 5 ml for e-EMR and 10 ml for ESD). All resection sites were photographed. Tattoos were placed to mark out at each most caudal and cranial sites.

Termination by intravenous pentobarbitone overdose and colectomy were then performed. Histological samples were taken to enable full thickness analysis of the bowel. Histology analysis were blindly assessed and reported to eliminate the selection bias. Submucosal depth and muscle viability were measured under a light microscope and photographed.

Although the invention has been particularly shown and described with reference to particular examples, it will be understood by those skilled in the art that various changes in the form and details may be made therein without departing from the scope of the present invention.

1. A composition for aiding the removal of polyps, in the form of a submucosal injectable formulation, the composition comprising one or more salts of hyaluronic acid, saline solution and indigo carmine, but not including a substance exhibiting an angiotonic effect.

2. A composition for aiding the removal of polyps, in the form of a submucosal injectable formulation, consisting essentially of, one or more salts of hyaluronic acid, saline solution and indigo carmine.

3. The composition according to claim 1, wherein the salt of hyaluronic acid is sodium.

4. The composition according to claim 1, wherein the hyaluronic acid is sodium hyaluronate in the range of 0.1 to 1% weight /volume (w/v) and indigo carmine is in the range of 0.1 to 1% weight/volume (w/v).

5. The composition according to claim 1, wherein the concentration of sodium hyaluronate is in the range of 0.24 to 0.40% weight /volume (w/v) and the concentration of indigo carmine is in the range of 0.001 to 1% weight /volume (w/v)w/v.

6. The composition according to claim 1, wherein the saline solution is 99.30% weight/volume (w/v), the sodium hyaluronate is 0.315% weight/volume (w/v) and indigo carmine is 0.009% weight/volume (w/v).

7. A method for removing a polyp from a mucous membrane, the method comprising:
injecting a composition of claim 1 into the polyp; and
resecting the polyp from the mucous membrane for biopsy.

8. The composition of claim 1 for use in endoscopic procedures.

9. The composition of claim 1 for use in endoscopic biopsy.

10. The composition of claim 1 for use in colonoscopy.

11. A kit for use in an endoscopic procedure, said kit comprising the composition according to claim 1, an endoscopic injection needle, a syringe, and instructions for use thereof.

12. The composition of claim 1, wherein the substance exhibiting an angiotonic effect is epinephrine, norepinephrine, or isoproterenol.

13. The composition according to claim 2, wherein the salt of hyaluronic acid is sodium.

14. The composition according to claim 2, wherein the hyaluronic acid is sodium hyaluronate in the range of 0.1 to 1% weight/volume (w/v) and indigo carmine is in the range of 0.1 to 1% weight/volume (w/v).

15. The composition according to claim 2, wherein the concentration of sodium hyaluronate is in the range of 0.24 to 0.40% weight/volume (w/v) and the concentration of indigo carmine is in the range of 0.001 to 1% weight/volume (w/v)w/v.

16. The composition according to claim 2, wherein the saline solution is 99.30% weight/volume (w/v), the sodium hyaluronate is 0.315% weight/volume (w/v) and indigo carmine is 0.009% weight/volume (w/v).
